(19) United States
(12) Patent Application Publication
(10) Pub. No.: US 2009/0104235 A1
(43) Pub. Date: Apr. 23, 2009

(54) Method for Producing of a Preparation of a Solid Dmso-Containing Silicone Oil Emulsion for the Binding of Reactive Oxygen Compounds in Human and Animal Bodies

(76) Inventor: Heinrich Exner, Mockern (DE)

Correspondence Address: PATENT CENTRAL LLC;Stephan A. Pendorf, 1401 Hollywood Boulevard, Hollywood, FL, 33020, US

(21) Appl. No.: 11/817,205

(22) PCT Filed: Feb. 24, 2006
(86) PCT No.: PCT/DE2006/000355

§ 371 Date: Nov. 21, 2007

(30) Foreign Application Priority Data
Feb. 25, 2005 (DE) 10 2005 009 515.1

(51) Int. Cl. A61K 9/00

(52) U.S. Cl. 424/400

(57) ABSTRACT

The invention relates to a method for production of an oral, rectal or vaginal preparation comprising a solid silicone oil emulsion, characterized in that said preparation comprises 0.01-85 wt. % dimethicone and 0.01-45 wt. % dimethylsulphoxide, whereby the preparation dissolves after application and the emulsion is released. The dimethylsulphoxide is then resorbed and the remaining components of the preparation remain in the gut or vagina as a result of the molecular size thereof and subsequently are totally deposited. The invention further relates to a preparation and a method for production of a preparation made from a silicone oil emulsion.

### FIELD OF THE INVENTION

A silicone oil emulsion is known from DE 103 13 196 for the prophylaxis and treatment of diseases following the formation of reactive oxygen radicals. This emulsion is given as a cream or as an injection. It comprises as key components 0.01-85 wt. % silicone, in particular, polydimethysiloxane, 0.01-35 wt. % hydrophobic emulsifying agent with an HLB value of 1 to 7 and/or hydrophilic emulsifying agent with an HLB value of 7 to 14 and/or 0.01-35 wt. % of a mixture of hydrophobic and hydrophilic emulsifying agent with an HLB value of between 1 and 14, 0.1-99 wt. % biocompatible saline solution and 0.01-40 wt. % dimethyl-sulphoxide. A disadvantage of the application as an injection, in particular with an intravenous injection, is that this may only be conducted by trained staff. This makes it necessary for the patient to visit a doctor or a hospital for each treatment. The application as a cream only has a localized effect, and is not particularly suitable for treatment of the entire body.

The background to the invention is as follows. Infections which result in inflammatory symptoms can lead to a collapse of the immune system—immune paralysis—(SIRS, or septic shock) due to the agglomeration of oxygen intermediates and their decay products. As a result of the use of the known emulsion in larger doses, a neutralisation of these unsaturated oxygen intermediaries is achieved, the immune paralysis is resolved and the phagocytosis of the pathogens can be continued.

Similar effects also occur when treating pain. The pain, which is caused by inflammations, represents the sensitive aspect of these unsaturated intermediates and their after-products. When the known emulsion is applied in higher doses, the pain is treated in its origins when the negative charges are buffered.

In connection with the treatment of diseases with the known silicone emulsion, the following observations have been made, which are based on physical-chemical properties: silicone oils are dielectric and dipolar. In the known emulsion, the silicone oils are emulsified and comprise a very large surface.

In most cases, a dielectric obtains its properties through polarisation. When a dielectric is brought into an electric field, the dipoles contained in the dielectric orient themselves according to the field. A dipole is generally considered to be the arrangement of two opposite charges—e.g. a molecule with an electrically positive and an electrically negative end. Due to this polarisation, the dielectric is charged, and stores energy which can be emitted again as soon as the electric field is removed.

The dipolar solvent which is present alongside silicone oil in the known emulsion and the emulsifying agents used, which are based on polyoxyethylene, increase the dipolar characteristic of the emulsion. The electrostatic reciprocal effects are produced, and so-called “Van der Waals forces”, which occur between permanent and induced dipoles, result. (D. Voet, Judith G. Voet, Charlotte W. Pratt, Biochemie p. 26 ff. 2002 Wiley VCH publishers, Weinheim).

The larger the surface of the dipolar silicone molecules, the larger the charge acceptance. In tests on rat paws, the removal of the silicone oils from the emulsion could already be observed from the first day onwards following the application of the emulsion. After the fourth day, the removal by means of monocytes and neutrophile granulocytes already decreased significantly.

The dipolar characteristic of the silicone oil which has not been emulsified is of no significance to the disintegration of pathogenic oxygen compounds due to its very low surface size.

As a result, only silicone oil emulsions are suitable for the transfer of electrons. The terminal restriction of the known silicone emulsions due to disintegration or metabolic processes of the components is of great significance with regard to their clinical effects.

The known silicone emulsion has already been described for the stimulation of the non-specific defence of the immune system in the event of a range of diseases. The dose in this case was 1 ml on average. This dose was sufficient according to the knowledge available at that time in order to be able to conduct a targeted treatment, and to activate the non-specific immune system. The first clinical effects during the treatment of the diseases not felt until between 1 and 3 weeks later.

The determination of the Van der Waals forces present in the known silicone emulsion opens an entirely new field for the use of this emulsion in the treatment of disease processes. The pathological effects which occur as a result of the unsaturated oxygen intermediates, together with their after-products, can be electrostatically buffered at an early stage, and offer a substantially better prognosis for the progress of the disease. The negative charges from the unsaturated oxygen intermediates are buffered by the known silicone emulsion. The neutralisation of these charges is achieved by the body itself.

The disadvantage with the type of application of this medication is that the silicone is absorbed by the body and can then be stored in tissues and organs. No disintegration of the silicone occurs. This is a significant disadvantage.

An adjuvant for antigens, together with a method for producing and applying an adjuvant for antigens, is known from the patent document DE 44 99 552. A further adjuvant emulsion is described in the WO document 02/074283.

The use of an oil-in-water emulsion as a medication or for the production of a medication is described in patent document U.S. Pat. No. 6,288,026.

A further oil-in-water emulsion for use as a medication or for the production of a medication is published in WO 00/50085.

The object of the invention is therefore to be able to provide a medication which is free of residues in order to exclude the possibility of inflammatory reactions occurring in the body.

Suppositories are also known from the prior art which contain substances and active substances which melt at body temperature. They usually take the form of cones, rolls or torpedoes, and are used in the rectal and/or vaginal application of active substances.

Active substances of this type can, according to EP 0 031 561 C1, comprise the following:

Xanthines, anti-cancer agents, antibiotics, polypeptides, antiarrhythmics, other cardiovascular agents, anti-diabetic agents, anti-ulcer agents, anti-fungal agents, sedatives, diuretics, anti-hypertensive agents and medications for the treatment of Parkinson's Disease.

In DE 37 09 861 A1, a silicone-free suppository basic mass is described, which consists of a non-soluble fatty mass and an additive product of between 5 and 50 Mol ethyl oxide on 1 Mol of hardened ricin oil as an emulsifying agent for the production of ampoules (suppositories) which contain aqueous active substance solutions. Preferably, between 0.2 and 10 wt. % of the emulsifying agent are contained. With the mass, silicone-free ampoules can be produced which contain up to 30 wt. % of dispersed water.

This makes it possible to incorporate active substances which are only available, or which can only be processed, in an aqueous solution. Active substances of this type can be processed in such quantities with the suppository basic masses that the ampoules which are obtained as a result contain up to 30 wt. % which is homogeneously distributed, i.e. homogeneously dispersed.

The background to the invention is the technical problem of providing a method for preparing a silicone emulsion for the treatment of infections (inflammations) which are caused by the agglomeration of oxygen intermediates and their after-products without additionally burdening and irritating the organism and immune system due to the storage of silicones.

In order to solve this problem, the invention provides a method for producing an oral, rectal or vaginal preparation which contains a solid silicone emulsion, characterized in that the preparation contains 0.01-85 wt. % silicone oil and 0.01-45 wt. % dimethylsulphoxide. The preparation stands out for the fact that it dissolves following application, and that the silicone emulsion is released, whereby dimethylsulphoxide (DMSO), together with any glycerine which has been added and/or the physiological saline solution, is reabsorbed, and the remaining components of the preparation remain in the bowel or the vagina due to their molecule size, and are then excreted without any residues remaining.

The background to the invention is the surprising discovery that the silicone emulsion which contains the DMSO remains stable in the bowel or in the vagina during and after the dissolution process of the fatty mass which is insoluble in water and which is contained in the preparation, and that as a result the large surface which is an absolute prerequisite for the buffering of the oxygen intermediates which damage the organism remains intact. It is also surprising that the effect of easing pain and restricting inflammation on the entire body (systemic effect) of the silicone emulsion which contains the DMSO remains, even though the silicone is not absorbed by the body, while the DMSO is reabsorbed by the body, however.

The effective mechanism of the preparation according to the invention is based on the fact that DMSO is released in the bowel in a continuous and even manner during the dissolution process of the preparation. There it is absorbed by the body via the mucous membrane in the bowel, and is transported within a few minutes to all organ systems via the blood circulation. In areas of organs which are at risk from inflammation, or areas with a high concentration of unsaturated oxygen intermediates, DMSO binds the oxygen intermediates and/or passes them on to the stable emulsion of the silicone oil in the bowel, where due to its high dipolar characteristic which is formed in the emulsion, the negative charges of the emulsified silicone are bound, neutralised and excreted via excrement. The reduction in pain is therefore felt within a very short period of time, in a maximum of 15 minutes. DMSO shows no depository effects in the body, since alongside the excretion via the bowel, it is also excreted via the skin and the lungs.

Literature: (Pharmacology of DMSO, Stanley W. Jacob and Robert Herschler, Department of Surgery, Oregon Health Science University, Portland, Oreg. 97201, 2001-2003).

It is found that the effect of DMSO according to the invention is only achieved in combination with the named silicone emulsion. A dose of pure DMSO or in combination with non-emulsified silicone does not lead to the high pain-relieving and inflammation-restricting effect according to the invention.

In addition, the preparation according to the invention which contains a solid silicone emulsion which contains DMSO guarantees that a slow and even emission of DMSO occurs from the preparation. On the one hand, this is due to the form of the emulsion per se, while on the other, it is due to the composition of the preparation. The silicone emulsion which contains DMSO is released in a slow and continuous manner when the fatty mass portion of the preparation which does not dissolve in water dissolves in the bowel or the vagina, i.e. the preparation according to the invention comprises alongside the depot characteristic of the silicone emulsion an additional depot characteristic. Due to the selection of the type and proportion of the components of the preparation, the speed of the releases of the silicone emulsion which contains DMSO can be determined. This can be achieved for example by the type of the selected fatty masses which do not dissolve in water (chain length, melting behaviour, binding characteristics). As a result, the dosage of the silicone emulsion which contains DMSO can be adapted to the nature of the disease which is to be treated.

As a result, significantly lower levels of DMSO can be applied with the same intensity of effect. With standard preparations for the treatment of chronic diseases among humans, DMSO can be prescribed in a dose of up to 1 g/kg of body weight. A toxic effect only occurs among humans when over 1 g/kg DMSO is supplied (N. M. Luerry et al. Drug. Intell. Clin. Pharm. 18, 591 [1984]). Due to the silicone emulsion according to the invention, the same effect is achieved among humans with an application quantity of DMSO of 1 mg/kg of body weight. With the silicone emulsion according to the invention, the quantity of DMSO to be applied is therefore reduced to 1/1000^{th} of the application quantity used to date.

An advantage of rectal application is that the primary liver passage is largely avoided, since the portions of the emulsion which are reabsorbed in the lower two-thirds of the rectum are transported directly into the lower vena cava and not into the portal vein. The effect of the silicone emulsion according to the invention is felt within several minutes, preferably between after 5 and 15 minutes, on average after 10 minutes, and is maintained sustainably over the entire resorption period of between 2 and 6 hours, on average, 4 hours.

Preferably, fatty masses which are insoluble in water which are used as the basic mass of the preparation according to the invention are selected from the group “cocoa butter, hardened vegetable fats, mixtures of fatty acids saturated with monoglyceride, diglyceride and triglyceride, waxes”.

The emulsifying agents according to the invention can be selected from the group “emulsifying agents with an HLB value of between 1 and 14”. Preferably, emulsifying agents based on polyoxyethylene are used. These increase the dipolar characteristic of the emulsion.

As further auxiliary substances, alongside cetylstearyl alcohol, beeswax, glycerine monostearate, aluminium stearate, bentonite, celluloses, fluid triglyceride, paraffins, highly dispersible silicic acids or siliceous earth, calcium carbonate, magnesium oxide and antioxidants can be added to the suppository according to the invention.

Preferably, the preparation consists of 0.01-85 wt. % silicone oils, in particular polydimethylsiloxane, 0.01-35 wt. % of hydrophobic emulsifying agent with an HLB value of between 1 and 7 and/or a hydrophilic emulsifying agent with an HLB value of between 7 and 14 and/or 0.01-35 wt. % of a mixture of hydrophobic and hydrophilic emulsifying agent with an HLB value of between 1 and 14, a 0.1-99 wt. % biocompatible saline solution, 0.01-40 wt. % dimethylsulphoxide (DMSO), 0.01-35 wt. % glycerine, 0.01-45 wt. % cetylstearyl alcohol, and 0.01-80 wt. % fatty mass which is insoluble in water, whereby the preparation dissolves following application and the emulsion is released, whereby dimethylsulphoxide (DMSO), glycerine and the physiological saline solution are reabsorbed, and the remaining components of the preparation remain in the bowel or the vagina due to their molecule size, and are then excreted with no residues remaining.

In a particular embodiment, the preparation consists of 8.3 wt. % dimethicone, 3.2 wt. % polysorbate 80, 11.5 wt. % cetylstearyl alcohol, 8.3 wt. % dimethylsulphoxide, 9.8 wt. % physiological saline solution, 9.8 wt. % glycerine, and 49.1 wt. % cocoa butter.

Preferably, the preparation according to the invention takes the form of a suppository or an oral application. The oral application according to the invention can be encased in a protective coating so that it does not already dissolve during its passage to the stomach or the small bowel. This protective layer is designed in such a manner that it only dissolves at the site of the application, e.g. the large bowel, and releases the solid silicone emulsion which contains DMSO there. Many protective layers of this type are known, and have been used for a long time for the protection of capsules, tablets or coated pills which are designed to reach the stomach or the bowel without losing their effect.

Preferably, the prescription which contains a solid silicone emulsion is used in the form of a suppository or an oral application for the treatment of pain conditions. In particular, pain conditions are treated which occur with diseases such as auto-immune diseases or neoplasias.

Furthermore, the solid silicone emulsion according to the invention has been successfully used in the form of a suppository in the treatment of blockages in the spinal area.

The preparation according to the invention is easy and painless in its application, as a result of which its acceptance among children and pain sufferers in particular will be significantly improved. Due to the form of the preparation as a suppository, it is possible to treat the entire body of the patient at home, since the patient can themselves apply the suppository or can have it applied by a member of the family, for example. With long-term therapies, fewer irritations are likely to occur than with repeated, in particular, muscular injections, since the silicone oils are excreted from the body after their dissolution in the bowel as an emulsion.

The silicone oil emulsion can be an oil-in-water or a water-in-oil emulsion, or a double emulsion.

In the large bowel, up to 90% of the substance transportation is conducted not through the enterocytes, but via the paracellular path. The tight junctions in the colon and the rectum are freely passable for molecules with a diameter of up to 0.2-0.25 mm, but are less passable or impassable for high molecular substances. An indirect scale used in order to determine the molecule size of silicone oil is the determination of the kinematic viscosity. It has been ascertained that the kinematic viscosity of the silicone oil must, according to PharmEUR, be at least 50 mm²·s⁻¹ in order to avoid resorption.

In a particular embodiment, the preparation according to the invention can be combined with water soluble and/or fat soluble active substances. Preferably, the preparation according to the invention can contain one or more water soluble active substances from the group “antibiotics, hydrogen peroxide, painkillers and water soluble vitamins”.

Equally, the preparation according to the invention makes it possible to apply fat soluble active substances. Fat soluble active substances can be fat soluble vitamins.

A particular aspect of the invention is the combination of one or more water soluble and one or more fat soluble active substances in one preparation.

In a particular embodiment, the preparation can be used for the treatment of genital diseases, whereby the preparation can also contain, in addition to the named active substances or as a single active substance, an aqueous hydrogen peroxide solution. The concentration of the hydrogen peroxide solution is here between 1 wt. % and 4.5 wt. %, preferably 2.5 wt. %. As a result, the treatment of vaginal mucous membrane diseases such as “candida infections, viral infections, and bacterial infections” is possible. The invention also provides a method for the production of a preparation according to the invention, comprising at least the following procedural stages: the heating of the entire recipe in a heating bath to 60-99° C., preferably 85° C., the homogenisation of the preparation mass with 12,000 to 14,000 rpm, preferably with 13,000 rpm, and the decanting of the preparation mass.

In a preferred embodiment, the preparation according to the invention is produced according to the following procedure:

All components of the preparation according to the invention are melted in a water bath and are heated to over 80° C. The homogenisation is then conducted using a stirring device with 12,000 rpm. The mass is thus turned to a paste and can then immediately be decanted into the cast moulds. In the refrigerator or at room temperature, it hardens within 12 hours.

An unanticipated positive effect of this method of production is that the solid preparation can be produced in a single stage. The silicone emulsion is stably formed in the simultaneous presence of the fatty mass which is insoluble in water. A further advantage of the method according to the invention is that the preparation mass cools down itself during homogenisation. An additional cooling procedure, which requires a large amount of energy, is therefore not necessary, as a result of which the method according to the invention has an economic advantage over known methods for the production of solid preparations for rectal, vaginal or oral application.

The invention will now be explained in greater detail with reference to preferred embodiments.

### EXAMPLES

H.S. * 1965, female: morbus Bechterev for over 10 years. Treatment of chronic pain with Celebrex. The disease progresses and can also not be significantly influenced by Celebrex. The side effects of Celebrex clearly cause difficulty to the patient. After a week of treatment with the suppository according to the invention, with daily application, the general condition already improved, and after 4 weeks of treatment, treatment with Celebrex could be terminated.

H.A. * 1964, female: mamma tumour diagnosed following OP, chemotherapy cycles and radiation.

The patient was given the suppositories according to the invention with daily application for improvement of quality of life. After several days, the pain in the joints caused by the therapy was already reduced, and the patient was again able to lead an active life.

R.B. * 1955, male: diagnosis: blockage of the vertebrae in the lumbar area. The patient had suffered a blockage in the lumbar area while laying a floor, and was no longer able to stand straight. Following the application of a suppository according to the invention, he was already able to continue his work.

S.J. * 1955, female: diagnosis: fatigue syndrome.

A patient with typical signs of exhaustion such as sleeplessness, inability to concentrate, sub-febrile temperature and general apathy. Following treatment with a suppository according to the invention, her sense of wellbeing quickly returned. Further treatment will be continued after a week if necessary.

P.K. * 1969, female: diagnosis: migraine attack.

For the treatment, two of the suppositories according to the invention were applied with an interim period of one hour. Afterwards, the patient was free of symptoms.

1. A method for producing an oral, rectal or vaginal preparation containing a solid silicone oil emulsion, characterized in that the preparation contains 0.01-85 wt. % dimethicone and 0.01-45 wt. % dimethylsulphoxide.

2. A method according to claim 1, characterized in that the preparation is formulated as a suppository or formulated for oral administration.

3. A method according to claim 1, characterized in that the preparation consists of
0.01-85 wt. % silicone,
0.01-35 wt. % hydrophobic emulsifying agent with an HLB value of between 1 and 7, and/or a hydrophilic emulsifying agent with an HLB value of between 7 and 14, and/or 0.01-35 wt. % of a mixture of hydrophobic and hydrophilic emulsifying agent with an HLB value of between 1 and 14,
0.1-99 wt. % biocompatible saline solution,
0.01-40 wt. % dimethylsulphoxide,
0.01-35 wt. % glycerine,
0.01-45 wt. % cetylstearyl alcohol, and
0.01-85 wt. % fatty mass which is insoluble in water.

4. A method according to claim 1, characterized in that the preparation consists of

| | | |
|---|---|---|
| 8.3 | wt. % | dimeticone, |
| 3.2 | wt. % | polysorbate 80, |
| 11.5 | wt. % | cetylstearyl alcohol, |
| 8.3 | wt. % | dimethylsulphoxide, |
| 9.8 | wt. % | physiological saline solution, |
| 9.8 | wt. % | glycerine, and |
| 49.1 | wt. % | cocoa butter. |

5. A method according to claim 1, characterized in that the dimethicone has a kinematic viscosity of greater than 50 mm²·s⁻¹.

6. A method according to claim 1, characterized in that the preparation is resorbed over a period of 2-6 hours.

7. A method according to claim 1, characterized in that the preparation is combined with water soluble and/or fat soluble active substances.

8. A method according to claim 7, characterized in that the water soluble active substances are selected from the group consisting of antibiotics, painkillers, vitamins, and hydrogen peroxide.

9. A method according to claim 7, characterized in that the fat soluble active substances are fat soluble vitamins.

10. The oral, rectal, or vaginal application of the preparation according to claim 13 for the treatment of auto-immune diseases, neoplasias, and diseases of the weight-bearing and musocskeletal systems.

11. The oral, rectal, or vaginal application of the preparation according to claim 13, for the treatment of genital diseases, whereby the preparation contains an aqueous hydrogen peroxide solution.

12. A method for producing a preparation according to claim 13, containing a solid silicone emulsion comprising at least the following procedural stages:
the heating of all components in a heating bath to 60-99° C.,
the homogenisation of the preparation mass with 10,000 to 15,000 rpm,
the decanting of the preparation mass.

13. A preparation containing a solid silicone emulsion, characterized in that the preparation contains 0.01-85 wt. % dimethicone and 0.01-45 wt. % dimethylsulphoxide.

14. A preparation according to claim 13, characterized in that the preparation consists of
0.01-85 wt. % silicone,
0.01-35 wt. % hydrophobic emulsifying agent with an HLB value of between 1 and 7 and/or a hydrophilic emulsifying agent with an HLB value of between 7 and 14 and/or 0.01-35 wt. % of a mixture of hydrophobic and hydrophilic emulsifying agents with an HLB value of between 1 and 14,
0.1-99 wt. % biocompatible saline solution,
0.01-40 wt. % dimethylsulphoxide,
0.01-35 wt. % glycerine,
0.01-45 wt. % cetylstearyl alcohol, and
0.01-85 wt. % fatty mass which is insoluble in water
characterized in that the silicone has a kinematic viscosity of over 50 mm²·s⁻¹.

15. A preparation according to claim 14, characterized in that the preparation is formulated as a suppository or formulated for oral administration.

16. The method according to claim 3, wherein the silicone is polydimethysiloxane.

17. The method according to claim 6, wherein the preparation is resorbed over a period of 4 hours.

18. The method according to claim 12, wherein the preparation mass is homogenized at 13,000 rpm.

19. The preparation according to claim 14, wherein the silicone is polydimethysiloxane.
